(19) United States
(12) Patent Application Publication
(10) Pub. No.: US 2024/0024132 A1
(43) Pub. Date: Jan. 25, 2024

(54) FEMORAL CONDYLE TRIAL IMPLANT

(71) Applicant: Aesculap AG, Tuttlingen (DE)

(72) Inventors: Franz-Peter Firmbach, Emmingen-Liptingen (DE); Svenja Anhorn, Heroldstatt (DE)

(21) Appl. No.: 18/348,620

(22) Filed: Jul. 7, 2023

(30) Foreign Application Priority Data
Jul. 25, 2022 (DE) 10 2022 207 580.3

(51) Int. Cl. A61F 2/46; A61F 2/38

(52) U.S. Cl. CPC A61F 2/4684; A61F 2/3859; A61F 2/461

(57) ABSTRACT

A femoral condyle trial implant for trial application to a resected femur in a knee joint replacement operation. The trial implant has a distal front face with a medial condyle surface, which is configured for articulation with a medial tibial plateau of a proximal tibia, and a lateral condyle surface, which is configured for articulation with a lateral tibial plateau of the proximal tibia. The trial implant also has a proximal rear face that is planar and forms a flat bone contact surface configured for contacting a distal cut surface of a distally resected femur.

### CROSS-REFERENCE TO RELATED APPLICATION(S)

This application claims priority under 35 U.S.C. § 119 to German Application No. 2022 207 580.3, filed on Jul. 25, 2022, the content of which is incorporated by reference herein in its entirety.

### FIELD

The present disclosure relates to a femoral condyle trial implant for trial application to a resected femur in a knee joint replacement operation.

### BACKGROUND

In a knee joint replacement operation, or total knee arthroplasty (TKA), joint surfaces of the femur and/or tibia that have become worn or have been otherwise affected by disease or by injury are replaced by artificial joint surfaces of a knee joint prothesis. Such knee joint prostheses usually comprise a femoral component and a tibial component. The femoral component is implanted at the distal end of the femur. The tibial component is implanted at the proximal end of the tibia.

Before the implantation of the prosthetic components, the distal femur and the proximal tibia are resected. For this purpose, the surgeon makes various resection cuts and detaches bone and/or cartilage material from the respective bone. The resection enables the respective bone to be adapted in its shape to the prosthetic component that is to be received.

The resection can be carried out on the basis of different concepts. One concept aims to keep the tension of the ligaments of the knee balanced during the movement of the joint. This is intended to ensure better functioning of the knee joint prosthesis. This concept is generally referred to as “gap balancing”. In other concepts, the surgeon uses the resection to remove a certain amount of bone and/or cartilage material. Such concepts are generally referred to as “measured resection”. The alignment of the resection cuts with respect to the patient's anatomy determines the subsequent alignment of the implanted components and consequently also the orientation of the prosthetic joint axes. The alignment of the resection cuts is therefore of particular importance.

In the alignment of the resection cuts, there are mainly three approaches: mechanical, anatomical and kinematic. Mechanical alignment involves resection of the proximal tibia perpendicularly to the longitudinal axis of the tibial shaft. The resection of the distal femur takes place in a manner correspondingly adapted thereto. If so required, ligament releases are performed. The anatomical alignment attempts to resect the tibia at a varus angle of 3°. Femoral resection and ligament releases are performed with the aim of obtaining a straight hip-knee-ankle axis of the leg. The goal of kinematic alignment is to implant the artificial joint surfaces of the prosthetic components at the level of the natural, defect-free joint surface present prior to the development of arthritis.

In kinematic alignment, the alignment of the resection cuts often starts on the distal femur. The resection of the proximal tibia is adapted thereto. In this context, this is also designated as a transfer of the alignments and/or cuts. For this purpose, special surgical instruments are known which are also designated as tibial cut alignment guides. Such instruments allow the alignment of the femoral resection cuts to be transferred to the tibia. The transfer usually takes place after an at least distal resection of the femur, in which the distal condyles are detached. The transfer can take place in extension or flexion.

A femoral trial implant is known from U.S. Pat. No. 10,912,658 B2 and is provided for use in an alignment of the tibial resection cuts. The known trial implant has a specially designed rear face which is configured to be applied to a completely resected femur. Completely means that the distal, anterior and posterior condyles are removed and additional chamfer cuts are made. A front face lying opposite the rear face of the trial implant has interfaces for releasable connection to a tibial transfer tool.

### SUMMARY

Against this background, there is a fundamental need for surgical instruments, trial implants and implants for implementation of kinematic alignment. Specifically, there is a need for instruments and trial implants that allow the alignment of the resection cuts to be transferred from the distal femur to the proximal tibia.

The object of the present disclosure is to permit improved transfer of the alignment of the resection cuts in the context of kinematic alignment.

The femoral condyle trial implant according to the present disclosure is provided for trial application to a resected femur in a knee joint replacement operation and has: a distal front face with a medial condyle surface, which is configured for articulation with a medial tibial plateau of a proximal tibia, and with a lateral condyle surface, which is configured for articulation with a lateral tibial plateau of the proximal tibia. Moreover, the femoral condyle trial implant according to the present disclosure has a proximal rear face, wherein the rear face is plane and forms a flat bone contact surface which is configured for contacting a distal cut surface of the distally resected femur. By virtue of the solution according to the present disclosure, the proximal tibial cut can be aligned before the anterior and posterior femoral cuts are made. For this purpose, the rear face of the femoral condyle trial implant is plane and forms said flat bone contact surface. The flat bone contact surface is configured for contacting the distal cut surface of the femur. The distal cut surface results from a distal femoral cut. In contrast to solutions known from the prior art, the present disclosure permits an improved alignment of the anterior and posterior femoral cuts. If so required, their alignment can also be fixed and/or corrected after the alignment of the tibial cut. This is not readily possible in solutions known from the prior art, since the anterior and posterior femoral cuts have to be made before the trial implant is applied and before the tibial cut is aligned on the basis of the latter. The two condyle surfaces of the femoral condyle trial implant replace the detached natural distal condyles of the femur. By contrast, the natural anterior and posterior femoral condyles are not replaced. The femoral condyle trial implant does not have any surfaces taking the place of the natural anterior and posterior femoral condyles. In the use of the femoral condyle trial implant, the natural anterior and posterior femoral condyles are usually not detached or not yet detached. In different embodiments, the trial application takes place in different ways, for example by cohesive bonding or force-fit and/or form-fit engagement.

The position and direction designations used in this description relate to the body of a patient, in particular the femur of the patient, and to this extent they are to be understood in accordance with their usual anatomical meaning. Consequently, “anterior” denotes front or lying to the front, “posterior” denotes rear or lying to the rear, “medial” denotes inner or lying to the inside, “lateral” denotes outer or lying to the outside, “proximal” denotes towards the centre of the body, and “distal” denotes away from the centre of the body. Furthermore, “proximodistal” denotes along, preferably parallel to, a proximal-distal axis, “anteroposterior” denotes along, preferably parallel to, an anterior-posterior axis, and “mediolateral” denotes along, preferably parallel to, a medial-lateral axis. The aforementioned axes are orthogonal to one another and can of course be understood in relation to X, Y and Z axes not associated with the anatomy of the patient. For example, the proximal-distal axis can be designated alternatively as the X axis. The medial-lateral axis can be designated as the Y axis. The anterior-posterior axis can be designated as the Z axis. For the sake of better illustration and simplicity of the designations, the aforementioned anatomical position and direction designations are primarily used in the following. Moreover, designations such as “front face” are used in relation to a proximally directed viewing direction. By contrast, designations such as “rear face” are used in relation to a distally directed viewing direction.

In one embodiment of the present disclosure, at least one fastening portion is present and is configured for releasable fastening to the distal femur. In one embodiment, the at least one fastening portion serves to receive a separate fastening element, for example a nail, a screw or the like. In a further embodiment, the at least one fastening portion itself forms such a fastening element. By virtue of the at least one fastening portion, it is possible in particular to dispense with a cohesively bonded placement of the femoral condyle trial implant. This can facilitate the use of the femoral condyle trial implant.

In one embodiment of the present disclosure, the at least one fastening portion is a through-bore which extends in a proximodistal direction between the front face and the rear face, and which is configured for receiving a cylindrical fastening pin. Starting from the front face, the cylindrical fastening pin can be introduced into the distal femur via the through-bore. For example, the cylindrical fastening pin can be a screw, a nail or the like. Preferably, at least two through-bores are present. In this case, a first through-bore is preferably arranged on the medial condyle surface and a second through-bore is arranged on the lateral condyle surface.

In a further embodiment of the present disclosure, the at least one fastening portion is a cylindrical fastening pin protruding from the rear face. In this embodiment, the femoral condyle trial implant for application to the resected femur can be pressed, with the cylindrical fastening pin to the front, onto the distal cut surface. In the process, the cylindrical fastening pin penetrates into the distally resected femur and brings about releasable force-fit fastening. The cylindrical fastening pin is preferably pointed at its proximal end. At least two cylindrical fastening pins are preferably present. In this case, a first cylindrical fastening pin is arranged to the rear of the medial condyle surface and a second cylindrical fastening pin is arranged to the rear of the lateral condyle surface. In this embodiment, it is possible to do without a separate fastening means. The fastening means, more precisely the cylindrical fastening pin, is an integral part and/or integrally moulded part of the femoral condyle trial implant.

In a further embodiment of the present disclosure, the front face has at least one first coupling portion, which is configured for releasable force-fit and/or form-fit coupling to a tibial alignment instrument for aligning a tibial cutting block. In the coupled state, the tibial alignment instrument is preferably positioned relative to the femoral condyle trial implant so as to be fixed and/or immovable. The femoral condyle trial implant, more precisely its alignment, serves as a reference for the alignment of the tibial cutting block and therefore also for the alignment of the tibial cut. The alignment is transferred from the femoral condyle trial implant to the tibial cutting block by means of the coupled tibial alignment instrument. In different embodiments, the at least one first coupling portion is designed differently, for example as a receiving bore, receiving cutout, receiving groove or the like. The releasable coupling can be a plug-in, latching, clamping and/or snap-fit connection. Preferably, at least two first coupling portions are present and are arranged spaced mediolaterally apart from each other on the front face.

In a further embodiment of the present disclosure, the at least one first coupling portion is a receiving bore introduced into the front face and extending in an anteroposterior direction. The anteroposterior longitudinal extent permits particularly simple coupling of the tibial alignment instrument. In one embodiment, the receiving bore is designed as a through-bore. In a further embodiment, the receiving bore is a blind hole open at one end and closed at the other end. In this case, the opening can be arranged anteriorly or posteriorly on the front face. The opening is preferably arranged anteriorly. Preferably, at least two receiving bores are present and are arranged spaced mediolaterally apart from each other on the front face.

In a further embodiment of the present disclosure, the front face has at least one second coupling portion which is configured for releasable force-fit and/or form-fit coupling to a compensation element for dimensional compensation of a defect of the tibial plateau. This is a particularly advantageous embodiment of the present disclosure. Defects of the tibial plateau, i.e. those caused by arthritic or other types of wear of the bones and/or cartilage, can easily be dimensionally compensated in the alignment of the tibial cut. For this purpose, the at least one second coupling portion is present on the front face and serves for releasable coupling to said compensation element. In the coupled state, the compensation element covers the medial condyle surface and/or the lateral condyle surface. In place of the respective condyle surface, the compensation element comes to bear on the relevant portion of the tibial plateau. In different embodiments, the releasable coupling is configured differently and/or the at least one second coupling portion is designed differently. The releasable coupling can be, for example, a plug-in, clamping, latching and/or snap-fit connection. Preferably, the front face has at least two second coupling portions, which are arranged spaced mediolaterally apart from each other. In this case, one of the two second coupling portions is preferably arranged on the medial condyle surface and another of the two second coupling portions is arranged on the lateral condyle surface.

The present disclosure further relates to an implant system for use in a knee joint replacement operation, having at least one femoral condyle trial implant according to the preceding embodiment and having at least one compensation element which is configured for releasable force-fit and/or form-fit coupling to the at least one second coupling portion of the femoral condyle trial implant and for dimensional compensation of a defect of the tibial plateau.

In a further embodiment of the present disclosure, a plurality of different compensation elements are present, wherein the different compensation elements have different proximodistal thicknesses for the dimensional compensation of defects of different extents. The different compensation elements can be applied to the front face of the femoral condyle trial implant interchangeably and according to the extent of an existing defect.

The present disclosure further relates to an implant system for use in a knee joint replacement operation, having a plurality of femoral condyle trial implants according to one of the preceding embodiments, wherein the plurality of femoral condyle trial implants are of different sizes and are configured for trial application to different sizes of femoral bones. In other words, in one embodiment the several femoral condyle trial implants differ in terms of a mediolateral dimension of the front face and/or the rear face. In a further embodiment, the several femoral condyle trial implants alternatively or additionally differ in terms of an anteroposterior dimension of the front face and/or rear face. In a further embodiment, the several femoral condyle trial implants alternatively or additionally differ in terms of a proximodistal thickness between the front face and the rear face.

### BRIEF DESCRIPTION OF THE DRAWINGS

Further advantages and features of the present disclosure will become clear from the claims and from the following description of preferred exemplary embodiments of the present disclosure which are shown in the drawings.

FIG. 1 shows, in a schematic representation and in a proximal viewing direction, an embodiment of a femoral condyle trial implant according to the present disclosure in a state when applied to a distal femur,

FIGS. 2 and 3 show different views of an example of an intraoperative situation using the femoral condyle trial implant, in a posterior viewing direction (FIG. 2) and in a medial viewing direction (FIG. 3),

FIG. 4 shows a further embodiment of a femoral condyle trial implant according to the present disclosure in a schematic and partially cutaway side view, in a medial viewing direction,

FIG. 5 shows a further embodiment of a femoral condyle trial implant according to the present disclosure in a schematic side view, in a medial viewing direction,

FIG. 6 shows a further embodiment of a femoral condyle trial implant according to the present disclosure in a schematic and partially cutaway side view, in a medial viewing direction,

FIG. 7 shows an embodiment of an implant system according to the present disclosure with a further embodiment of a femoral condyle trial implant according to the present disclosure and with a plurality of different compensation elements, the implant system being shown in a schematic and partially cutaway side view, in a medial viewing direction, and

FIG. 8 shows a further embodiment of an implant system according to the present disclosure with a plurality of different femoral condyle trial implants, the implant system being shown schematically and in a proximal viewing direction.

### DETAILED DESCRIPTION

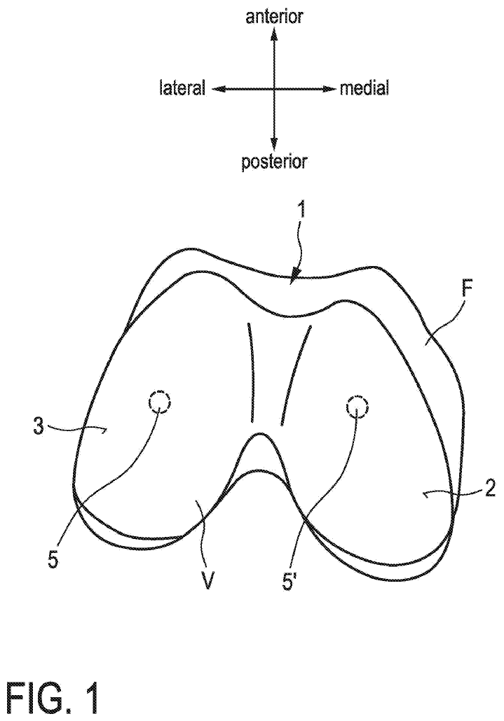

According to FIGS. 1 to 3, a femoral condyle trial implant 1 is provided for use in a knee joint replacement operation. The femoral condyle trial implant 1 is referred to below for short as the condyle implant.

The condyle implant 1 is configured for trial application to a distal femur F and has a distal front face C and a rear face R lying proximally opposite the front face V.

In the use of the condyle implant 1, the rear face R is oriented proximally and faces towards the distal femur F. The front face V is oriented distally and faces towards a proximal tibia, more precisely the tibial plateau TP of the latter.

The front face V has a medial condyle surface 2 and a lateral condyle surface 3. The medial condyle surface 2 is configured for articulation with a medial portion of the tibial plateau TP. This portion can also be designated as the medial tibial plateau TPL. The lateral condyle surface 3 is configured for articulation with a lateral portion of the tibial plateau TP. This portion of the tibial plateau TP can also be designated as the lateral tibial plateau TPL.

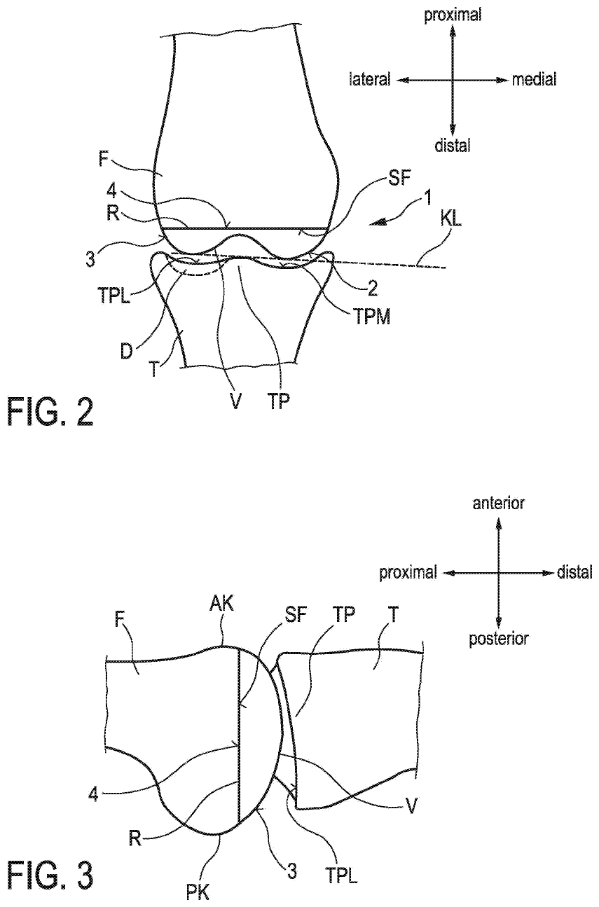

The rear face R is plane and forms a flat bone contact surface 4. The flat bone contact surface 4 is configured for contacting a distal cut surface SF of the distally resected femur F. In the intraoperative situation shown in FIGS. 1 to 3, the two condyle surfaces 2, 3 of the condyle implant 1 replace the natural distal condyle surfaces of the femur F. Before application of the condyle implant, the latter surfaces were detached by means of a distal femoral cut in a manner known to a person skilled in the art. The femur F still has its anterior condyle surfaces AK and its posterior condyle surfaces PK. In contrast to the natural distal condyle surfaces, the anterior condyle surfaces AK and the posterior condyle surfaces PK are not replaced by means of the condyle implant 1. The two condyle surfaces 2, 3 simulate a distal condyle line KL of the femur (see FIG. 2).

The medial condyle surface 2 and the distal condyle surface 3 are curved convexly and, in their shape, they simulate a shape of the natural distal condyle of the femur F. It will be appreciated that the shape of the condyle surfaces 2, 3 shown in the figures is to be seen purely as an example.

The condyle implant 1 is applied to the femur 1 on a trial basis. It is applied in different ways in different embodiments. In an embodiment not shown in the figures, a cohesively bonded connection is provided. For this purpose, the rear face R can be joined cohesively to the distal cut surface SF. Medical adhesives or the like that are suitable for this purpose are known to a person skilled in the art.

In the embodiments shown in the figures, a releasable force-fit fastening is provided. For this purpose, the condyle implant 1 has at least one fastening portion 5. The fastening portion 5 is differently designed in different embodiments.

In the embodiment shown, the condyle implant 1 has two fastening portions 5, 5′ (see FIG. 1). The two fastening portions 5, 5′ are arranged spaced mediolaterally apart from each other and can also be designated as lateral fastening portion 5 and medial fastening portion 5′. Apart from their different arrangement, the two fastening portions 5, 5′ are of identical design. Therefore, in order to avoid repetition, further details of the fastening portions 5, 5′ are explained below with reference to only one of the two fastening portions 5, 5′. What is said about it applies to both fastening portions 5, 5′.

In the embodiment shown, the condyle implant 1 has a one-piece design. A design with several parts (multi-piece design) is also conceivable. The materials used are metals and plastics that are suitable for medical purposes. Such materials are known to a person skilled in the art.

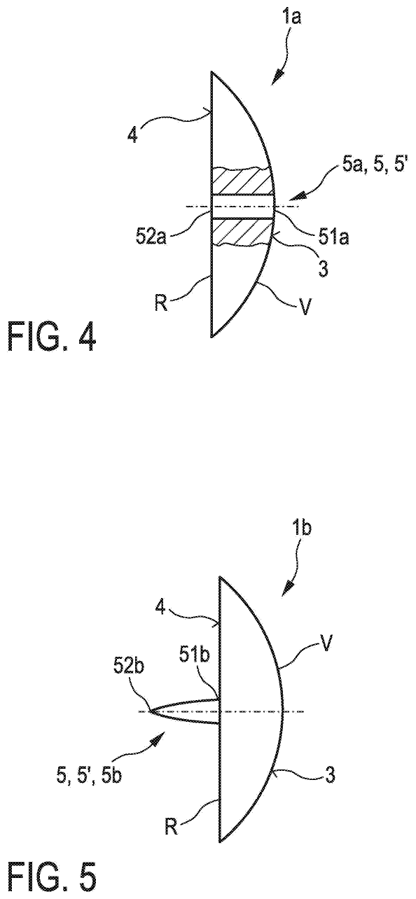

FIGS. 4 to 7 show further embodiments of femoral condyle trial implants 1a, 1b, 1c, 1d according to the present disclosure. The design and function of the condyle implants 1a, 1b, 1c, 1d are in principle identical to the design and function of the condyle implant 1 according to FIGS. 1 to 3. Therefore, in order to avoid repetition, only essential differences of the condyle implants 1a, 1b, 1c, 1d are explained below. Parts having the same function are not described in each embodiment. Instead, reference is made expressly to what has been said concerning the condyle implant 1 according to FIGS. 1 to 3.

In the condyle implant 1a according to FIG. 4, the fastening portion 5 is a through-bore 5a. The through-bore 5a extends continuously between the front face V and the rear face R. The through-bore 5a has a distal opening 51a arranged on the front face V and a proximal opening 52a arranged on the rear face R. The through-bore 5a extends along a proximodistal axis through the condyle implant 1a. The through-bore 5a is configured to receive a cylindrical fastening pin (not shown in the figures). Such fastening pins are known to a person skilled in the art and can be designed, for example, as a screw, nail, peg or the like. For the trial application of the condyle implant 1a to the distal femur F, the rear face R is brought into contact with the distal cut surface SF. Thereafter, said cylindrical fastening pin, starting from the front face V, is inserted through the distal opening 51a into the through-bore 5a and introduced into the distal cut surface SF via the proximal opening 52a.

In the condyle implant 1b according to FIG. 5, the fastening portion 5 is a cylindrical fastening pin 5b. The fastening pin 5b protrudes in the proximal direction from the rear face R. The fastening pin 5b has a distal end 51b and a proximal end 52b. The fastening pin 5b extends between the distal end 51b and the proximal end 52b. The distal end 51b is rigidly connected to the rear face R. The proximal end 52b is pointed in the present case. In the embodiment shown, the fastening pin 5b is in integral with the other portions of the condyle implant 1b. In an embodiment not shown in the figures, the fastening pin is a separately manufactured component which is subsequently joined together with the rear face. For the trial application of the condyle implant 1, the rear face R is positioned opposite to the distal cut face F and the fastening pin 5b is pressed manually into the bone structure of the femur F.

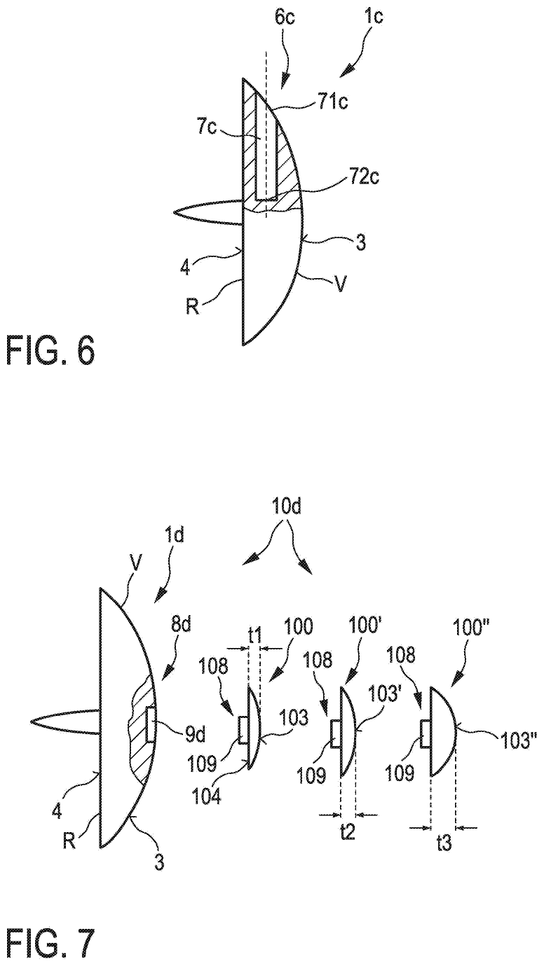

In the condyle implant 1c according to FIG. 6, a first coupling portion 6c is present. The first coupling portion 6c serves for releasable force-fit and/or form-fit coupling to a tibial alignment instrument for aligning a tibial cutting block. Such tibial alignment instruments are known to a person skilled in the art and serve to transfer the orientation of the distal cut surface SF to the proximal tibia T. The first coupling portion 6c is arranged on the front face V. In the embodiment shown, the first coupling portion 6c is a receiving bore 7c. The receiving bore 7c extends in the anteroposterior direction and in the present case has an anterior opening 71c and a posterior stop 72c. The receiving bore 7c extends between the opening 71c and the stop 72c and in this sense is designed as a blind hole. The receiving bore 7c is configured to receive a complementary coupling pin of said tibial alignment instrument. The condyle implant 1c in the present case has a further first coupling portion (not visible in FIG. 6). This further coupling portion is spaced medially from the first coupling portion 6c shown in FIG. 6 and is arranged in the region of the medial condyle surface 2.

The condyle implant 1d according to FIG. 7 has at least one second coupling portion 8d. The second coupling portion 8d is configured for releasable force-fit and/or form-fit coupling to a compensation element 100. The compensation element 100 serves for dimensional compensation of a defect D of the tibial plateau TP (see FIG. 2). Together with the at least one compensation element 100, the condyle implant 1d forms an implant system 10d.

In the embodiment shown, the second coupling portion 8d is arranged in the region of the lateral condyle surface 3. In the present case, the condyle implant 1d has a further second coupling portion, which is not visible in FIG. 7. This further second coupling portion is arranged in the region of the medial condyle surface 2.

For releasable coupling to the condyle implant 1d, the compensation element 100 has a complementary coupling portion 108. The complementary coupling portion 108 is arranged on a rear face of the compensation element 100.

In the embodiment shown, the second coupling portion 8d is a latching receptacle 9d. Accordingly, the complementary coupling portion 108 of the compensation element 100 is a complementary latching portion 109. The latching receptacle 9d is recessed in the proximal direction into the front face V. The complementary latching portion 109 protrudes proximally from the rear face of the compensation element 100.

The compensation element 100 has a distal contact surface 103 and a proximal rear surface 104. The contact surface 103 is arranged on a front face of the compensation element. The contact surface 103 is configured for articulation with the tibial plateau TP. The contact surface 103 is convex. The contact surface 103 is smaller than the condyle surfaces 2, 3. Depending on whether the compensation element 100 is mounted laterally or medially on the front face V of the condyle implant 1, the contact surface 103 makes contact with the medial tibial plateau TPM or the lateral tibial plateau TPL. In the mounted state, the rear surface 104 bears on the front face V of the condyle implant 1d.

In the embodiment shown, the implant system 10d has a plurality of different compensation elements 100, 100′, 100″. These are also designated as first compensation element 100, second compensation element 100′ and third compensation element 100″.

The plurality of compensation elements 100, 100′, 100″ are interchangeably connectable to the condyle implant 1d. Accordingly, the second and third compensation elements 100′, 100″ each have a coupling portion/latching portion 108, 109, which is identical to the coupling portion 108/latching portion 109 of the first compensation element 100.

The compensation elements 100, 100′, 100″ differ in terms of a proximodistal thickness t1, t2, t3 and serve for dimensional compensation of defects of different extents. Depending on the extent of the defect, a comparatively thin or a comparatively thick compensation element is used.

Specifically, the first compensation element 100 has a first thickness t1. The second compensation element 100′ has a second thickness t2. The third compensation element 100″ has a third thickness t3. In the present case, the first thickness t1 is less than the second thickness t2 and the third thickness t3. The second thickness t2 is greater than the first thickness t1 and less than the third thickness t3. The third thickness t3 is greater than the first thickness t1 and the second thickness t2.

In the embodiment shown, an external diameter (not defined in detail) of the compensation elements 100, 100′, 100″ is identical. On account of the different thicknesses t1, t2, t3, the respective contact surfaces 103, 103′, 103″ have different curvatures. Alternatively, compensation elements with different external diameters are conceivable.

It will be appreciated that the shown number of compensation elements is purely an example. The implant system 10d can of course have more or fewer than the shown three compensation elements 100, 100′, 100″.

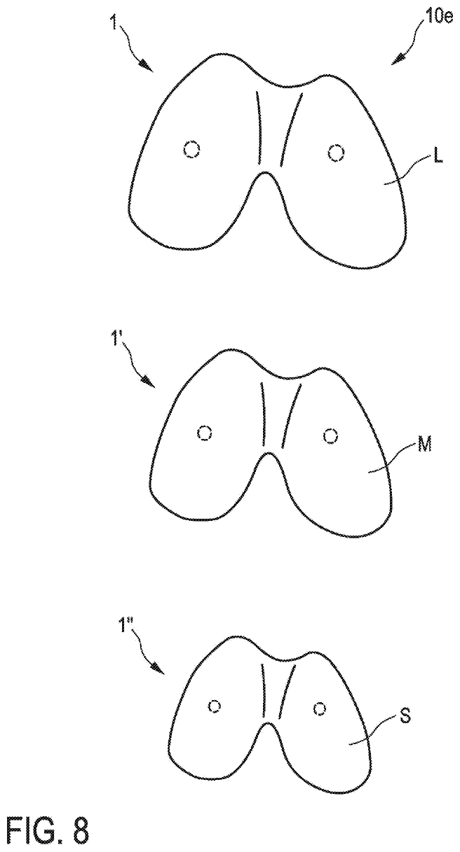

FIG. 8 shows a further embodiment of an implant system 10e according to the present disclosure. The implant system 10e comprises a plurality of different femoral condyle trial implants 1, 1′, 1″, which are also designated below as first condyle implant 1, second condyle implant 1′ and third condyle implant 1″. The different condyle implants 1, 1′, 1″ have different sizes L, M, S. The different sizes of condyle implants 1, 1′, 1″ are provided for trial application to different sizes of femoral bones.

In the embodiment shown, the condyle implants 1, 1′, 1″ differ in terms of their mediolateral and/or anteroposterior dimensions. Moreover, the proximodistal dimensions of the condyle implants 1, 1′, 1″ are different, although this is not discernible in FIG. 8. The sizes L, M, S can also be designated as large, medium and small.

The features of the condyle implants 1, 1a, 1b, 1c and 1d can be combined with one another in any desired manner to give further combinations of features. The same applies analogously to the features of the implant system 10d, 10e. For example, a condyle implant according to the present disclosure, not shown in the figures, has at least one first coupling portion and a second coupling portion. It is also understood that, other than shown in the figures, the fastening portions of the condyle implants 1c, 1d can be designed in each case as a through-bore. Moreover, an implant system according to the present disclosure, not shown in the figures, has, on the one hand, condyle implants of different sizes and, on the other hand, a plurality of different compensation elements, which can be optionally arranged on each of the different condyle implants.

1. A femoral condyle trial implant for trial application to a resected femur in a knee joint replacement operation, the femoral condyle trial implant comprising:
a front face comprising a medial condyle surface configured for articulation with a medial tibial plateau of a proximal tibia, and comprising a lateral condyle surface configured for articulation with a lateral tibial plateau of the proximal tibia; and
a rear face;
the rear face being planar and forming a flat bone contact surface configured for contacting a distal cut surface of the resected femur.

2. The femoral condyle trial implant according to claim 1 further comprising at least one fastening portion configured for releasable fastening to the resected femur.

3. The femoral condyle trial implant according to claim 2, wherein the at least one fastening portion is a through-bore which extends in a proximodistal direction between the front face and the rear face and which is configured for receiving a cylindrical fastening pin.

4. The femoral condyle trial implant according to claim 2, wherein the at least one fastening portion is a cylindrical fastening pin protruding from the rear face.

5. The femoral condyle trial implant according to claim 1, wherein the front face has at least one first coupling portion configured for releasable force-fit and/or form-fit coupling to a tibial alignment instrument for aligning a tibial cutting block.

6. The femoral condyle trial implant according to claim 5, wherein the at least one first coupling portion is a receiving bore introduced into the front face and extending in an anteroposterior direction.

7. The femoral condyle trial implant according to claim 1, wherein the front face has at least one second coupling portion configured for releasable force-fit and/or form-fit coupling to a compensation element for dimensional compensation of a defect of a tibial plateau.

8. An implant system comprising:
at least one femoral condyle trial implant according to claim 7; and
the at least one compensation element.

9. The implant system according to claim 8 further comprising a plurality of different compensation elements, wherein the different compensation elements have different proximodistal thicknesses for dimensional compensation of defects of different extents.

10. An implant system comprising a plurality of femoral condyle trial implants according to claim 1, wherein the plurality of femoral condyle trial implants are of different sizes and are configured for trial application to different sizes of femoral bones.
